(19) United States
(12) Patent Application Publication
(10) Pub. No.: US 2009/0281394 A1
(43) Pub. Date: Nov. 12, 2009

(54) Bio-mechanical sensor system

(76) Inventors: Brian Keith Russell, Papakura (NZ); Stephen Christopher Kent, Auckland (NZ); Paul Benjamin Mallinson, Auckland (NZ); Christopher Michael Solomon, Auckland (NZ); Nicholas Alistair Close, North Shore City (NZ)

Correspondence Address: JACOBSON HOLMAN PLLC, 400 SEVENTH STREET N.W., SUITE 600, WASHINGTON, DC, 20004, US

(21) Appl. No.: 12/311,276

(22) PCT Filed: Sep. 21, 2007
(86) PCT No.: PCT/NZ2007/000277

§ 371 Date: Jun. 25, 2009

(30) Foreign Application Priority Data
Sep. 25, 2006 (AU) 2006905273

(51) Int. Cl. A61B 5/00; A61B 5/04

(52) U.S. Cl. 600/301; 600/388

(57) ABSTRACT

The bio mechanical sensor system is disclosed that uses conductive fabric sensors to detect, monitor and record one or more physiological parameters of a person wearing a garment that incorporates the fabric sensors such as a body harness or strap for example, that is attached to a person. The physiological parameters that can be detected include a wearer's heart rate and respiration rate plus ambient temperature and body temperature for example. The garment has a monitoring device that is attached to the garment and used to receive the detected physiological data. A processing circuit within the monitoring device then processes the data and outputs the person's physiological data to a display device in a format characteristic of the person's heart rate and respiratory rate and/or outputs the data to a third party system for review and analysis.

### TECHNICAL FIELD

The present invention relates to a system and method for monitoring multiple bio mechanical parameters of an individual. More particularly, the present invention relates to a personal bio mechanical harness system that uses conductive fabric sensors to detect a number physiological parameters for review and/or analysis using a third party system.

### BACKGROUND ART

The monitoring of an individual's physiological parameters is a routine process in clinics and hospitals. Such monitoring generally requires the individual to lie down and have a number of adhesive type patches attached to a patient's chest area for example. Each of the patches are connected to monitoring equipment using electrical cables or leads enabling the individual's physiological parameters to be monitored, recorded and analysed for diagnostic purposes. It is generally preferable to attach up to 12 patches and associated leads to the individual in order to monitor and assess a patient's condition. A similar sort of system is used for monitoring a patient's electrocardiogram (ECG), requiring separate monitoring equipment to be used as well as separate patches and cables to be attached to the individual. As such, this type of physiological monitoring system is not practical for use in a portable mode and would be impractical to wear over a long period of time or to be used as a monitoring and analysis tool for individual's undertaking outdoor exercise type activities.

U.S. Pat. No. 6,783,493 to VivoMetrics Incorporated discloses an apparatus and method for extracting a cardiac signal from a plethysmographic signal which is responsive to at least one cardiac parameter using a non invasive monitoring technique. Electrocardiograph electrodes are attached to or embedded within a garment worn by the person being monitored. The garment provides a “close fit” to the patient's skin enabling the sensors to detect the expansion and contraction of the patient's chest for example, as they breathe. Because the cardiac signals of interest generally have a small signal amplitude and are usually obscured by considerably larger amplitude respiratory or other undesired signals, careful processing is required to extract useful cardiac information from the received signal. Furthermore, although this device has a number of systems incorporated within the jacket worn by the patient, this type of system is not conducive to athletes wishing to monitor their heart rate and/or ECG for a prolonged period due to the relative bulk of the jacket worn by the user.

It is an object of the present invention to provide improved bio-mechanical sensing system for detecting and measuring a person's physiological data, or at least to provide industry or the public with a useful choice.

In this specification if reference has been made to patent specifications, other external documents, or other sources of information, this is generally for the purpose of providing a context for discussing the features of the invention. Unless it is specifically stated otherwise, reference to such external documents is not to be construed as an admission that such documents, or such sources of information, in any jurisdiction, are prior art or form part of the common general knowledge in the art.

### DISCLOSURE OF THE INVENTION

Accordingly, in a first aspect the present invention is said to consist in a bio-mechanical sensing system comprising:

a plurality of conductive fabric sensors integral with a garment and capable of sensing physiological data in an analogue signal format,

a plurality of connectors integral with said garment,

a monitoring device electrically connected to each of said plurality of conductive fabric sensors by at least one of said plurality of connectors, said monitoring device including:
a detection circuit used to receive as an input said physiological signal data detected by said plurality of fabric sensors, and
a processing circuit for processing and extrapolating said received physiological signal data into a readable digital data format characteristic of said physiological signal data.

Preferably, said monitoring device further includes a storage device for storing said digital data and a communications system for communicating said digital data to a third party system for analysis and/or storage of said digital data.

Preferably, said monitoring device further includes a user interface for outputting said digital data to a visual and/or audible output device for analysis and/or review by an individual.

Preferably, said plurality of conductive fabric sensors are used to sense a heart rate signal data and a respiratory rate signal data.

Alternatively, said plurality of conductive fabric sensors are used to sense a heart rate signal data and a respiratory rate signal data said bio-mechanical sensing system including at least one or more alternative sensor selected from the list comprising: a pressure sensor, a temperature sensor, a direction sensor or a movement sensor.

Preferably, said plurality of connectors are capable of carrying one or both of said heart rate signal data and said respiratory rate signal data detected by said plurality of conductive fabric sensors via said electrical connection to said monitoring device.

Preferably, said plurality of connectors are capable of carrying one or both of said heart rate signal data and said respiratory rate signal data detected by said plurality of conductive fabric sensors plus one or more of said alternative sensor inputs via said electrical connection to said monitoring device.

Preferably, at least one of said plurality of conductive fabric sensors is locatable such that said at least one conductive fabric sensor is a respiratory rate sensor.

Preferably, said plurality of conductive fabric sensors are formed of a material to provide a plurality of compression capacitive sensors.

Preferably, said plurality of compression capacitive sensors provide a means for measuring an amount of compression between a wearer's body and said garment.

Preferably, said compression capacitive sensors are each formed by at least an upper and a lower layer of conductive fabric material and having an inner layer therebetween of compressible non-conductive material and provides a means for measuring a change in a separation distance between an upper and a lower layer of each of said plurality of conductive fabric sensors.

Preferably, said monitoring device generates an AC signal.

Preferably, said AC signal varies with a wearer's thoracic or diaphragm diameter when said wearer inhales.

Preferably, at least one of said plurality of conductive fabric sensors is a capacitive fabric compression sensor formed by at least an inner layer and an outer layer of conductive fabric having a compressible non-conductive material between each of said layers of conductive fabric.

Preferably, said compressible non-conductive material deforms or compresses due to a wearer's thoracic or diaphragm diameter changing when said wearer inhales thereby decreasing a separation distance between said inner and said outer layer of said conductive fabric.

Preferably, at least one of said plurality of conductive fabric sensors is locatable such that said at least one conductive fabric sensor is a respiratory rate sensor and said respiratory rate sensor has a first terminal coupled to an AC ground within said monitoring device and a second terminal coupled to an AC signal having a high output resistance such that said respiratory rate sensor has an output characteristic equivalent to that provided by a variable capacitor.

Preferably, said change in thoracic or diaphragm diameter causes said AC signal to change in amplitude due to a change in capacitive coupling between said inner and said outer layer of conductive fabric due to said change in said separation distance.

Preferably, said processing circuit samples the amplitude of said AC signal to generate a digital respiration related waveform and data representative of said wearer's respiration rate for storage in a storage device within said monitoring device and/or for output to a third party device.

Preferably, at least two of said plurality of conductive fabric sensors includes an electrical pad attached to or integral with one surface of each of said at least one of said plurality of conductive fabric sensors.

Preferably, said electrical pad abuts said wearer's skin surface and is electrically connected to said monitoring device.

Preferably, at least two of said plurality of conductive fabric sensors is locatable on said wearer's skin surface in a position such that said at least two of said plurality of conductive fabric sensors provide a heart rate sensing system.

Preferably, said processing circuit measures a change in a voltage between at least two of said electrical pads located on each side of an wearer's chest to generate a digital heart rate related waveform representative of said wearer's heart rate for storage in a storage device within said monitoring device and/or for output to a third party device.

Preferably, said compressible non-conductive material is constructed of an open cell foam type material.

Preferably, said layers of conductive fabric are formed from a stretchable and flexible fabric material.

Preferably, said plurality of conductive fabric sensors are substantially elastic enabling said plurality of conductive fabric sensors to stretch and conform to a wearer's body shape.

Preferably, said garment is selectable from the list comprising: a stretchable body harness or strap, a jacket type garment, a protective armour garment and an item of clothing for wearing on the upper body part of said user.

Preferably, said monitoring device includes a communications system and said communications system is a radio transmitter.

Alternatively, said monitoring device includes a communications system and said communications system includes a communications port.

Preferably, said communications port includes a wireless transmitter.

Alternatively, said communications port provides a user interface between a third party system and said monitoring device enabling said third party system to download said physiological signal data from said monitoring device to said third party system.

Preferably, said plurality of connectors provides a snap-fit type connection with said monitoring device.

Preferably, said plurality of connectors provide a serial interface connection between said garment and a third party system when said monitoring device is removed from said garment.

Preferably, said electrical connection to at least one of said plurality of connectors is made by at least one conductive thread.

Preferably, said monitoring device is a low power battery driven device.

In a second aspect the invention is said to consist in a garment used to sense a wearer's heart rate and respiratory rate comprising:

a stretchable harness system capable of attachment around a wearer's body using an attachment means,

a plurality of conductive fabric sensors integral with said stretchable harness system,

a plurality of connectors integral with said stretchable harness, and

a monitoring device electrically connected to each of said plurality of conductive fabric sensors by at least one of said plurality of connectors, said monitoring device including:
a detection circuit used to receive as an input a wearer's sensed heart rate signal and a wearer's respiratory rate signal detected by said plurality of fabric sensors, and
a processing circuit for processing and extrapolating said received heart rate and respiratory rate signals and processing said wearer's sensed heart rate signal and a wearer's respiratory rate signal into a digital signal data format characteristic of said wearer's sensed heart rate signal and a wearer's respiratory rate signal.

Preferably, said monitoring device further includes a storage device for storing said digital data and a communications system for communicating said digital data to a third party system for analysis and/or storage of said digital data.

Preferably, said monitoring device further includes a user interface for outputting said digital data to a visual and/or audible output device for analysis and/or review by an individual.

Alternatively, said plurality of conductive fabric sensors are used to sense a heart rate signal data and a respiratory rate signal data.

Preferably, said plurality of conductive fabric sensors are used to sense a heart rate signal data and a respiratory rate signal data said bio-mechanical sensing system including at least one or more alternative sensor selected from the list comprising: a pressure sensor, a temperature sensor, a direction sensor or a movement sensor.

Preferably, said plurality of connectors are capable of carrying one or both of said heart rate signal data and said respiratory rate signal data detected by said plurality of conductive fabric sensors via said electrical connection to said monitoring device.

Alternatively, said plurality of connectors are capable of carrying one or both of said heart rate signal data and said respiratory rate signal data detected by said plurality of conductive fabric sensors plus one or more of said alternative sensor inputs via said electrical connection to said monitoring device.

Preferably, at least one of said plurality of conductive fabric sensors is locatable such that said at least one conductive fabric sensor is a respiratory rate sensor.

Preferably, said plurality of conductive fabric sensors are formed of a material to provide a plurality of compression capacitive sensors.

Preferably, said plurality of compression capacitive sensors provide a means for measuring an amount of compression between a wearer's body and said garment.

Preferably, said compression capacitive sensors are each formed by at least an upper and a lower layer of conductive fabric material and having an inner layer therebetween of compressible non-conductive material and provides a means for measuring a change in a separation distance between said upper and said lower layer of each of said plurality of conductive fabric sensors.

Preferably, said monitoring device generates an AC signal.

Preferably, said AC signal varies with the wearer's thoracic or diaphragm diameter when said wearer inhales.

Preferably, at least one of said plurality of conductive fabric sensors is a capacitive fabric compression sensor formed by at least an inner layer and an outer layer of conductive fabric having a compressible non-conductive material between each of said layers of conductive fabric.

Preferably, said compressible non-conductive material deforms or compresses due to a wearer's thoracic or diaphragm diameter changing when said wearer inhales thereby decreasing a separation distance between said inner and said outer layer of said conductive fabric.

Preferably, at least one of said plurality of conductive fabric sensors is locatable such that said at least one conductive fabric sensor is a respiratory rate sensor and said respiratory rate sensor has a first terminal coupled to an AC ground within said monitoring device and a second terminal coupled to an AC signal having a high output resistance such that said respiratory rate sensor has an output characteristic equivalent to that provided by a variable capacitor.

Preferably, said change in thoracic or diaphragm diameter causes said AC signal to change in amplitude due to a change in capacitive coupling between said inner and said outer layer of conductive fabric due to said change in said separation distance.

Preferably, said processing circuit samples the amplitude of said AC signal to generate a digital respiration related waveform and data representative of said wearer's respiration rate for storage in a storage device within said monitoring device and/or for output to a third party device.

Preferably, at least two of said plurality of conductive fabric sensors includes an electrical pad attached to or integral with one surface of each of said at least one of said plurality of conductive fabric sensors.

Preferably, said electrical pad abuts said wearer's skin surface and electrically connected to said monitoring device.

Preferably, at least two of said plurality of conductive fabric sensors is locatable on a wearer's skin surface in a position such that said at least two of said plurality of conductive fabric sensors provide a heart rate sensing system.

Preferably, said processing circuit measures a change in a voltage between at least two of said electrical pads located on each side of a wearer's chest to generate a digital heart rate related waveform representative of a wearer's heart rate for storage in a storage device within said monitoring device and/or for output to a third party device.

Preferably, said compressible non-conductive material is constructed of an open cell foam type material.

Preferably, said layers of conductive fabric are formed from a stretchable and flexible fabric material.

Preferably, said plurality of conductive fabric sensors are substantially elastic enabling said plurality of conductive fabric sensors to stretch and conform to a wearer's body shape.

Preferably, said stretchable harness system is attached to or integral with and selectable from the list comprising: a body harness or strap, a torso band, a jacket type garment, a protective armour garment and an item of clothing for wearing on the upper body part of said user.

Preferably, said monitoring device includes a communications system and said communications system is a radio transmitter.

Alternatively, said monitoring device includes a communications system and said communications system includes a communications port.

Preferably, said communications port includes a wireless transmitter.

Alternatively, said communications port provides a user interface between a third party system and said monitoring device enabling said third party system to download a wearer's sensed heart rate signal data and respiratory rate signal data from said monitoring device to said third party system.

Preferably, said plurality of connectors provides a snap-fit type connection with said monitoring device.

Preferably, said plurality of connectors provide a serial interface connection between said stretchable harness system and a third party system when said monitoring device is removed from said stretchable harness system.

Preferably, said electrical connection to at least one of said plurality of connectors is made by at least one conductive thread.

Preferably, said monitoring device is a low power battery driven device.

Preferably, said attachment means is selectable from the list including: a Velcro strap type of attachment, a hook and eye type attachment, a snap-fit type of attachment or a press-fit type attachment.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

The term ‘comprising’ as used in this specification and claims means ‘consisting at least in part of’, that is to say when interpreting statements in this specification and claims which include that term, the features, prefaced by that term in each statement, all need to be present but other features can also be present.

The invention consists in the foregoing and also envisages constructions of which the following gives examples only.

### BRIEF DESCRIPTION OF THE DRAWINGS

The present invention will now be described with reference to the accompanying drawings in which:

FIG. 1 is a block diagram of the bio-mechanical sensor system of the present invention.

FIG. 2 is a sectional view and top view of the bio-mechanical harness used in the bio-mechanical sensor system of FIG. 1.

FIG. 3 is a cross-sectional view of the conductive fabric sensor using a compression sensor mechanism as applied to the bio-mechanical harness of FIG. 2.

FIG. 4 is a cross-sectional view of the conductive fabric sensor using a stretchable capacitive sensor mechanism as applied to the bio-mechanical harness of FIG. 2.

FIG. 5 is a graphic output of a user's heart rate and respiratory rate on a third party system that has been downloaded from the bio-mechanical sensor system of FIG. 1.

FIG. 6 is a block diagram of the electronic circuit for measuring a user's heart rate as applied to the bio-mechanical sensor system of FIG. 1.

FIG. 7 is a block diagram of the electronic circuit for measuring a user's respiratory rate as applied to the bio-mechanical sensor system of FIG. 1.

### DESCRIPTION OF THE PREFERRED EMBODIMENTS OF THE INVENTION

The bio mechanical sensor system of the present invention can be used in ambulatory monitoring, emergency room situations, in the home or even be used whilst exercising. The system provides a means of sensing, monitoring and recording an individual's physiological parameters such as their heart rate, respiration rate, ambient temperature and body temperature using a wearable garment such as a single body harness or strap attached to the individual. The list of sensor types that can be incorporated into the body harness may also include non-physiological sensors such as pressure sensors for sensing altitude changes, a flux gate compass for sensing an individual's direction of travel or an accelerometer to measure angle of orientation and activity. Whilst a number of sensing mechanisms have been mentioned these are in no way limiting as the sensing system can be used in a wide variety of environments and for a wide variety of purposes.

### Conductive Fabric Sensors

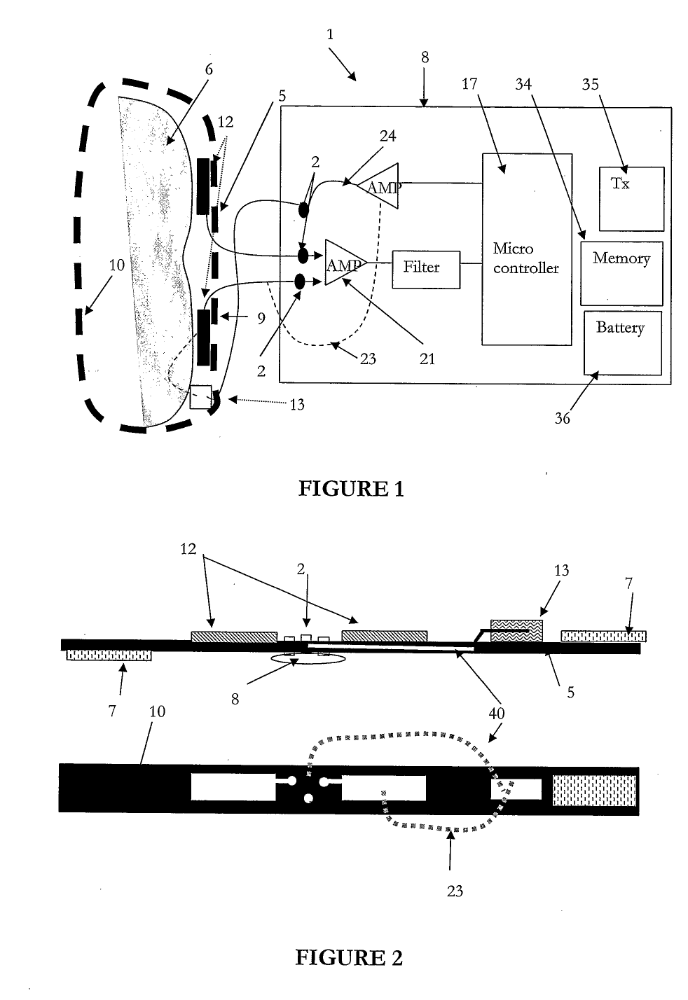

A preferred embodiment of the bio mechanical sensing system 1 of the present invention is shown in FIG. 1. The sensing device 1 preferably combines the measurement of both ECG and respiration through a shared connector scheme 2 using a number of conductive fabric sensors 12, 13 integral with a wearable garment 5 such as a body harness, torso band, jacket type garment or even protective armour. A preferred form of the wearable garment 5 is shown in FIG. 2. The wearable garment 5 is preferably a harness or strap type configuration that attaches around the circumference of the individual user's chest area 6. The strap 5 has two ends that are attached to each other to form a band using an attachment mechanism 7 such as Velcro attachments, press-fit or snap-fit type attachments. The electronic sensor processing and monitoring device 8 is attached using preferably three snap-fit type connectors 2 that are integral with a portion of the strap 5, preferably in close proximity to the user's sternum when the strap 5 is attached around the user's chest 6. These connections 2 provide an electrical connection between the conductive fabric sensors 12, 13 and the electronic processing device 8 using an internal conductive fabric connection 40. Whilst it is preferable to use three snap-fit electrical connectors 2, the use of two connectors may also be used. Furthermore, the system 1 can use four connectors, two connectors can carry heart rate/ECG signal data whilst the second two connectors can carry respiratory rate signal data. The electronic processing device 8 is attached to the front surface 9 of the strap 5 whereas the conductive fabric sensors 12 are located on the opposite (rear) surface 10 of the strap 5 such that they are in contact with the user's chest 6.

It has been found that the use of three electrical connectors 2 is preferable as the weight of the strap or band 5 is reduced and provides increased comfort for the wearer. Furthermore, the design of the conductive fabric sensors 12, 13 is such that signal interference between the ECG waveform and movement of the individual from muscle nerves and skin-sensor resistance changes is limited.

### Respiration Measurement

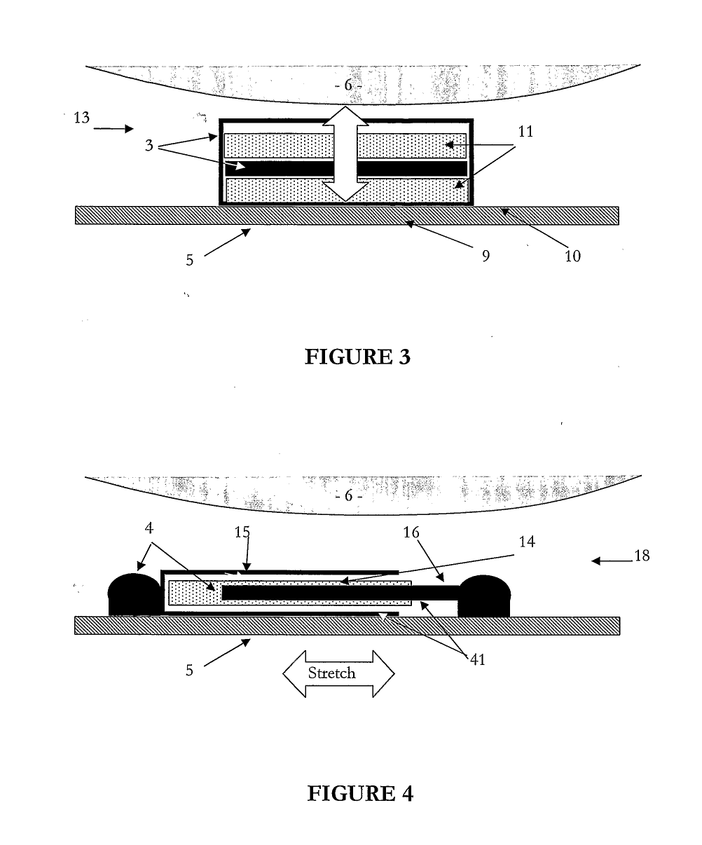

The conductor fabric sensors 13 are provided in a layered configuration within or on the surface of the strap or garment 5 as shown in FIGS. 3 and 4. Between each layer of conductive fabric 3, is a compressible non-conductive material 11 such as open cell polyethelene foam. This configuration provides a fabric compression sensor 13. The compression sensor 13 is manufactured to be an integral part of the wearable garment 5 and is configured such that the conductive fabric sensor 13 is flexible, formable and made from a stretchable elastic type material. The compression of the foam 11 in the fabric sensor material is less than the elasticity of the material used for the construction of the remainder of the wearable garment 5. For example, were the conductive fabric sensors 13, 18 are integral with a strap or band 5, the conductive sensors 13, 18 are located within the band 5 at strategic positions within the band 5 to enable the individual's respiration rate, for example, to be monitored. The remainder of the band 5 located around the wearers body or chest 6 is made of an elastic material that is less elastic than the conductive fabric sensor material 13, 18 to ensure the band 5 is comfortable when worn.

With reference to the compression fabric sensor 13 as shown in FIG. 3, when the individual inhales, their diaphragm expands forcing the compression sensors 12 to change shape by compressing the conductive fabric 3 and compressible material 11 thereby decreasing the distance between the fabric layers 3 as a result of the increased diameter of the individual's diaphragm. The amount of compression sensor 13 deformation is constrained by the elasticity of the band 5 around the individual's chest 6. Hence, the change in compression between the conductive fabric and compressible material layers 3, 11 causes a change in the electric field coupling (capacitance) between the conductive fabric layers 3 that can be detected and measured by the electronic sensing and monitoring device 8 connected to the band 5. This type of system is a compression capacitive sensing system 13.

Whilst compression sensors are preferred, it is also possible to measure respiration by sensing variations in the individual's bio mechanical status by using a layered conductive fabric sensing system 18 that is layered with a non-compressible material 14 located between each of the conductive fabric sensor layers 4 as shown in FIG. 4. For this type of fabric sensor it is preferable to have sensor fabric layers 4 grouped into two sets of sensing layers 15, 16 allowing for the capacitive sense signal variation between two plates or layers 41 to be determined. One end of the sensor fabric layer set is attached or fixed to the elastic strap or band (for example) and the second fabric layer set 16 has one end fixed at one end of the band 5 with the inner ends of each fabric layer set 15 being “unattached” and therefore moveable with respect to each other. For this type of configuration, the sensor system 18 measures the stretch of the band 5 during inhalation by measuring the variation in electric field coupling (capacitance) between each of the two sensor sets 15, 16 at either end of the conductive fabric layer 4. This type of system is a stretchable capacitive sensing system 18.

The strap or band 5 of the present invention can incorporate both the compression and stretchable capacitive sensors 13, 18 within a single strap or band 5. The portable monitoring and sensing device 8, interfaced to the sensors 13, 18 via the electrical connectors 2 on the strap 5, then uses electronic circuitry within the monitoring device 8 and the microcontroller 17 applies a number of algorithms to discriminate and measure the individual's bio-mechanical parameters sensed by the each of the sensor types 13, 18.

Whilst it is preferable that the non-conductive materials 4, 11 used between the conductive fabric layers 3, 4 are made of a stretchable type material, they can alternatively be made of a non-stretchable material. If non-stretchable material is to be used it is necessary to use elastic attachments to each of the conductive fabric layers 3, 4 to provide a means of constricting the fabric sensors 13, 18 back to their original shape and configuration when the individual exhales thereby decreasing their thoracic diameter. As such, the compression capacitive sensing system 13 measures the compression between the individual's chest or body 6 and the material chest strap or band 5. The stretchable capacitive sensing system 18 on the other hand uses conductive fabric layers 4 and measures the overlap of the conductive fabric sensor surfaces which are the distances.

### Sensor Processing and Monitoring Device

Each of the fabric sensors 12, 13 located within the wearable garment 5 are electrically connected to an electronic sensor processing and monitoring device 8 by a number of connectors 2 as discussed above. It is general common knowledge that due to the signal characteristics and noise carried within ECG/respiratory rate type signals it is preferable to utilise separate bio-mechanical sensors and cables when measuring an individual's ECG and heart rate. The present invention however, provides processing and electronic circuitry within the sensor processing device 8 that enables preferably two or three electrical connectors 2 to be used for carrying an individual's ECG and respiratory rate signals from the fabric sensors 12, 13 to the processing device 8 without interference between the signals. Hence the device shares connectors 2 for both ECG and respiratory signals.

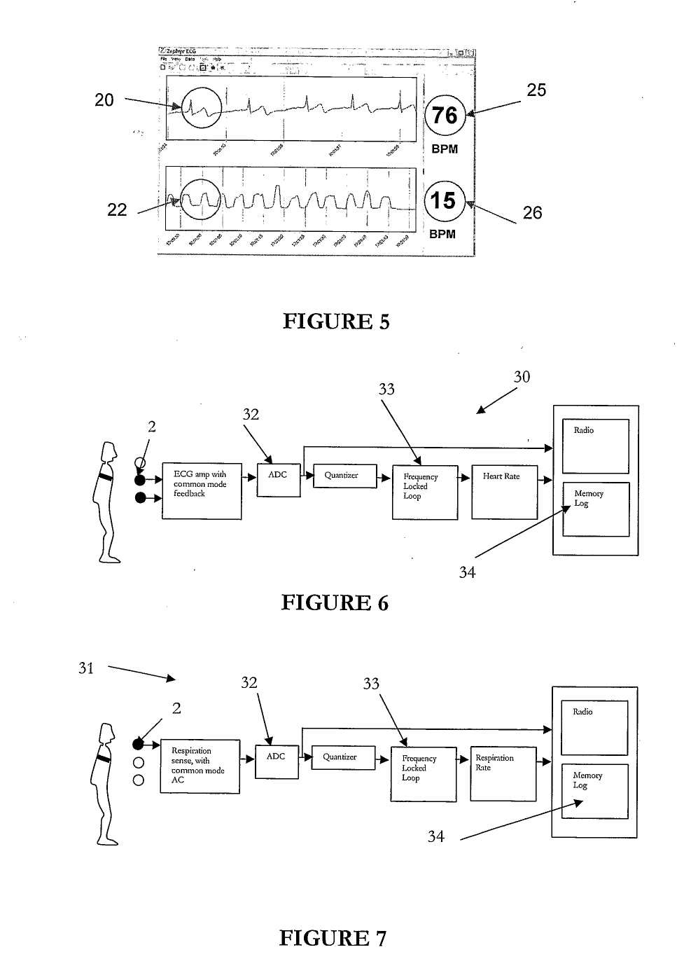

The electronic processing and monitoring device 8 provides a circuit that can be used to measure respiration as a result of increased pressure between conductive fabric layers 4 during inhalation whilst at the same time measuring the user's skin voltage using the compression sensors 12, due to cardiac response. As an ECG signal 20, as shown in FIG. 5, is a low frequency signal, the ECG amplifier 21 exhibits a low pass frequency response. The breathing/respiratory rate sensor 13 uses an AC signal generated by the processing device 8 and driven within a controlled high output resistance. Thus a breathing rate signal 22 is of a higher frequency than the ECG circuit sensitivity and as such is ignored by the ECG circuit as shown in FIG. 6. The breathing rate sensor 13 acts as a variable capacitor which has one terminal coupled to AC ground 23 and the other terminal attached to the high impedance drive signal 24. The AC ground 23 is the common mode point of the ECG signal coupled through the skin or a resistor (not shown) within the monitoring device 8. As a result, the respiration circuit of FIGS. 1 and 7 only needs one connector 2 compared with the ECG's two connectors 2 as the ECG circuit acts as the respiration sensor to ground. The AC signal between the high impedance AC signal and the variable capacitor varies with the thoracic or diaphragm expansion during inhalation. The AC signal amplitude is sampled by the microcontroller 17 within the processing device 8 to provide the respiration related waveform 22. A typical output of the ECG waveform 20 and corresponding heart rate in numerical form 25 is shown in FIG. 5 whereby the ECG frequency range is in the order of 150 Hz whilst the capacitive or respiratory rate sensing is performed at a frequency in the range of 33 kHz shown by the different respiratory waveform 22 and corresponding respiratory rate in numerical form 26. The AC ground of the respiration circuit 31 can be achieved by using the conductive path of the user's skin 23 as a connection to the ECG circuit 30. This enables a minimum number of connectors 2 to be used enabling a smaller, lighter and less obtrusive device 8 to be attached to the strap 5.

The heart rate sensors 12 use standard ECG type signals to measure the voltage across the chest 5. Two conductive fabric patches 12 with integral electric pads are placed on the individual such that the electric pads abut the individual's skin surface. These pads are used to measure heart rate and positioned with one on the front left of the chest 6 and one on the front tight hand side of the chest 5. A third sensor 13 is placed to the side of the chest 6 to measure respiration. This third sensor 13 can be combined within the ECG sensors 12. It has been found that one of the problems with this type of device is that any form of mechanical movement generates a large noise signal. During active exercise the ECG signal can include increased noise signals due to the variation in sensor-skin contact as well as other issues associated with the movement of the individual. However, it has been found that using the conductive fabric configuration of the present invention overcomes a substantial amount of the noise signal generated. Furthermore, by adding further padding between the fabric sensor 12, 13 and the elastic body band 5 to which the fabric sensors 12, 13 are attached has enabled movement aberrations to be isolated.

The processing device 8 is a low power device that is powered by batteries 36 and can be switched on using a manual switch (not shown) located on the device 8. Alternatively, the device 8 can be turned on automatically when the processing device 8 receives respiratory signals (when the individual puts on the wearable garment and breathes), by sensing skin conductivity or even by sensing the individual's movement. Each of these “turn-on” configurations can be set at the time of manufacture or alternatively at a later stage using the third party system and software to interface with the portable electronic sensing device 8.

The circuit uses a differential amplifier with feedback to filter out any input noise signals whilst at the same time detecting a pulse signal from the ECG waveform. This signal is then converted by the analogue to digital converter (ADC) 32 before being processed by the microcontroller 17. Respiration sensing is performed by using the microcontroller crystal (not shown) to provide a sinewave reference source signal and driving one of the conductive fabric sensing layers through a large resistance, such as 100 kOhms, whilst the remainder of the conductive fabric sensing layers are connected to the AC ground. Hence, the change in the conductive fabric sensing layer capacitance will alter the peak to peak sinewave signal input to the resistor. This sinewave signal provides an input to the microcontroller 17 to drive the microcontroller 17 and to enable synchronous sampling to be undertaken by the on-board ADC 32. A software algorithm residing within the microcontroller 17 performs peak to peak analysis on the received sinewave signal input to remove any DC signals which will occur due to initial garment fitting and ECG related noise. Once these signals and other sensor inputs are determined the rate (either pulses per minute or breaths pet minute) is extrapolated and calculated by a software frequency locked loop (FLL) 33. The loop response of the FLL 33 can have rules to allow for high signal to noise ratios and periods were no signal is received thereby giving a continuous rate output. These numbers are either stored in memory 34 within the processing device 8 for transmission or communication via the transmitter 35 to a third party system (not shown) at a later date. Alternatively, the data can transmitted, preferably wirelessly using a radio transmitting device 35, in real time to a scientific medical instrument or other third party device to be logged and the received data analysed. This can be achieved by the user interfacing with the sensor processing device 8 over communications link 35 using a software program residing in the third party system to activate the data download from the device 8 to the third party system to enable the third party system to be used to configure, view and analyse the bio-mechanical data. Alternatively, the interfacing is achieved by using the electrical connectors 2 used to attach the sensor monitoring device 8 to the strap or band 5. When the device 8 is removed from the band 5, the connectors 2 can be used for serial communication with a third party system (not shown). Additionally, the same connectors 2 can be used to charge an internal rechargeable battery 36.

### Data Processing Circuitry

FIGS. 6 and 7 show a block diagram of the processing circuitry 30, 31 used to receive and extract heart rate and respiration rate data 25, 26 respectively from the signal inputs received from the conductive fabric sensors 12, 13. The circuitry 30, 31 is driven by the microcontroller software using time domain filtering techniques coupled with frequency locked loop computational algorithms to convert analogue heart rate and respiratory signals into useable and meaningful digital formats for storage and/or output to a third party system. The third party system (not shown) subsequently converts the digital data to a numerical and graphic output display device for analysis and review by a medical professional for example.

### Optional Features

The bio-mechanical sensor system 1 is used to monitor heart rate and respiratory rates of the user. However, as already mentioned, other sensors can be incorporated into the wearable garment 5. Examples of sensors and features that can be monitored are as follows: Movement measurement devices such as solid state accelerometers, solid state gyroscopes, mechanical vibration switches or piezo-electric movement detectors.

A temperature sensor to measure ambient and body temperature such as thermistor or infra-red pickup semiconductor device. This device is thermally coupled to one of the electrical connectors as this is the best position to pick up skin temperature.

Pressure sensors to measure altitude using devices such as piezo bridges.

A flux gate compass for direction sensing.

A combination of sensors such as compass and pressure based altitude plus accelerometer based cadence can be used to dead reckon the distance and height traveled by the individual.

Multiple compression sensors can be used and can be positioned on the left and right side of the individual's torso to measure left-right differences. This type of system would provide feedback on the difference between the left and right sides of the body when an individual has suffered a stroke for example.

Multiple wearable sensor garments or bands can be worn vertically down the individual's torso to determine for example upper (apical verses lower (diaphragm) breathing differences and patterns.

The device can use the movement of the body and the compression of the band to extract energy and contribute to the power supply of the device. A device such as a magneto device, piezo-mass device or similar can be used to achieve this type of functionality.

The device can use the thermal difference between the body to generate power. This can be by way of semiconductor device located within the sensor processing device having the correct properties or thermal cycle engines alternatively heating and cooling.

The low power bio-mechanical sensing system 1 of the present invention provides a lightweight wearable device that can be used in a broad range of environments and conditions to provide information and feedback on a number of the user's physiological parameters. The portability and usability of the device in a broad range of environments has been achieved by providing a system that shares connectors 2 through which at least two different sensed signals 20, 22 can be carried coupled with a processing system that is capable of discriminating between these signals to provide a digital output indicative of the sensed signals.

1-71. (canceled)

72. A bio-mechanical sensing system comprising:
a plurality of conductive fabric sensors integral with a garment and positioned in the garment to contact the chest of the wearer when the garment is worn, to sense physiological data of a wearer, including two sensors for providing an analogue heart rate signal and a sensor for providing an analogue respiratory signal,
a plurality of electrical connectors integral with said garment including a first connector from one of the two heart rate sensors, a second connector from the respiratory rate sensor, and a third and shared connector from the other of the two heart rate sensors and which provides a relative ground for the respiratory rate signal,
a monitoring device electrically connected or connectable to said sensors by said electrical connectors including said shared connector, said monitoring device including:
a detection circuit arranged to receive as an input said analogue physiological signals from said fabric sensors, and
a processing circuit for processing and extrapolating said signals into a readable digital data format characteristic of said physiological signal data.

73. The bio-mechanical sensing system according to claim 72 wherein said monitoring device further includes a storage device for storing said digital data and a communications system for communicating said digital data to a third party system for analysis and/or storage of said digital data.

74. The bio-mechanical sensing system according to claim 72 wherein said monitoring device further includes a user interface for outputting said digital data to a visual and/or audible output device for analysis and/or review by an individual.

75. The bio-mechanical sensing system according to claim 72 wherein said plurality of conductive fabric sensors also include one or more of a pressure sensor, a temperature sensor, a direction sensor and a movement sensor.

76. The bio-mechanical sensing system according to claim 72 wherein each of said electrical connectors is a press-fit or snap-fit connector or hook and loop connector.

77. The bio-mechanical sensing system according to claim 72 wherein said respiratory rate sensor is formed by at least an inner and an outer layer of conductive fabric material having a compressible non-conductive material between and arranged to deform or compress due to a wearer's thoracic or diaphragm diameter changing when said wearer inhales thereby decreasing a separation distance between said inner and said outer layer of said conductive fabric.

78. The bio-mechanical sensing system according to claim 77 wherein said respiratory rate sensor has a first terminal coupled to an AC relative ground within said monitoring device by said third and shared electrical connector and a second terminal coupled by said second electrical connector to an AC signal driven with a high output resistance.

79. The bio-mechanical sensing system according to claim 78 wherein said processing circuit is arranged to sample the amplitude of said AC signal to generate a digital respiration related waveform and data representative of said wearer's respiration rate for storage in a storage device within said monitoring device and/or for output to a third party device.

80. The bio-mechanical sensing system according to claim 79 wherein said processing circuit is arranged to measure a change in a voltage between said heart rate sensors, to generate a digital heart rate related waveform representative of said wearer's heart rate for storage in a storage device within said monitoring device and/or for output to a third party device.

81. The bio-mechanical sensing system according to claim 80 wherein said processing circuit comprises an ECG amplifier with a low pass frequency response to exclude a higher frequency AC respiration rate signal.

82. The bio-mechanical sensing system according to claim 72 wherein said conductive fabric sensors are flexible for conforming to a wearer's body shape.

83. The bio-mechanical sensing system according to claim 72 wherein said garment is a stretchable body harness or strap, a jacket type garment, a protective armour garment, or other item of clothing for wearing on the upper body part of said user.

84. The bio-mechanical sensing system according to claim 72 wherein said monitoring device includes a communications port comprising a wireless transmitter.

85. The bio-mechanical sensing system according to claim 72 wherein said plurality of electrical connectors provide a serial interface connection between said garment and a third party system when said monitoring device is removed from said garment.

86. The body harness or strap, jacket, protective armour garment, or other item of clothing including a part arranged to contact a wearer's chest when worn, and comprising a biochemical sensing system according to claim 72.

87. A bio-mechanical sensing system comprising:
a body harness or strap or clothing item comprising integral conductive fabric positioned in the body harness or strap or clothing item to contact the chest of a wearer when worn, the conductive fabric sensors including two sensors for obtaining an analogue heart rate signal and a sensor for obtaining an analogue respiratory signal, the body harness or strap or clothing item comprising a first integral electrical connection from one of the heart rate sensors, a second integral electrical connection from the respiratory rate sensor, and a third integral electrical connection from both the other heart rate sensor and the respiratory rate sensor, and
a monitoring device electrically connected or connectable to said sensors and arranged to measure a change in a voltage between said heart rate sensors to provide a heart rate signal and comprising an ECG amplifier with a low pass frequency response to exclude a higher frequency AC respiration rate signal, the respiratory rate sensor being a capacitance-based sensor and the monitoring device coupling a first side of the respiratory rate sensor to an AC signal driven with a high output resistance and by said shared electrical connection a second side the respiratory rate sensor to an AC relative ground within said monitoring device.
